(19) United States
(12) Patent Application Publication
(10) Pub. No.: US 2025/0177733 A1
(43) Pub. Date: Jun. 5, 2025

(54) INSTRUMENTED COCHLEAR IMPLANT

(71) Applicant: CARNEGIE MELLON UNIVERSITY, Pittsburgh, PA (US)

(72) Inventors: Maysamreza Chamanzar, Pittsburgh, PA (US); Jay Reddy, Pittsburgh, PA (US)

(73) Assignee: CARNEGIE MELLON UNIVERSITY, Pittsburgh, PA (US)

(21) Appl. No.: 18/843,490

(22) PCT Filed: Mar. 28, 2023
(86) PCT No.: PCT/US2023/016517

§ 371 Date: Sep. 3, 2024

Related U.S. Application Data
(60) Provisional application No. 63/324,839, filed on Mar. 29, 2022.
(60) Provisional application No. 63/324,871, filed on Mar. 29, 2022.

(51) Int. Cl. A61N 1/05; A61N 1/36; A61N 1/372

(52) U.S. Cl. CPC A61N 1/0541; A61N 1/36038; A61N 1/372

(57) ABSTRACT

Disclosed herein is a novel design of an instrumented cochlear implant, wherein the electrode array portion of the implant is provided with a microfabricated thin-film sensing array comprising one or more sensors to detect various features of the electrode array during insertion and to provide feedback to the surgeon during implantation. The sensing array collects intraoperative information on the state of the electrode array during insertion.

### RELATED APPLICATIONS

This application claims the benefit of U.S. Provisional Patent Application Nos. 63/324,839 and 63,324,871, both filed Mar. 29, 2022, the contents of which are incorporated herein in their entireties.

### BACKGROUND

When hearing loss progresses beyond the point where hearing aids are effective, acoustic hearing can be augmented with electronic hearing via a cochlear implant. These life-changing devices supplement acoustic hearing with electronic hearing by directly stimulating the auditory nerve.

FIG. 1 shows an in situ cochlear implant. As can be seen, the cochlea is a spiral-shaped structure through which an electrode array portion of the implant must be placed. The electrode array is threaded into the scala tympani chamber of the cochlea via a small hole drilled in the base of the cochlea. The electrode array consists of a plurality of electrodes, typically composed of platinum, embedded in a substrate, typically silicone. Although it varies from manufacturer to manufacturer, the electrode array may be approximately 0.6 mm in diameter and 2-3 cm in length. The electrode array is flexible to conform to the anatomy of the cochlea during insertion.

The electrode arrays are difficult to insert consistently. During insertion, the electrode tip can apply excessive forces to the inner surface of the scala tympani, destroying the fragile hair cells. Other adverse events include tip foldover or piercing the basilar membrane, resulting in scalar translocation. These surgical events can reduce the performance of the implant, as well as causing trauma and thereby destroying or diminishing any remaining acoustic hearing capability of the patient. Additionally, the precise placement of the cochlear implant in the scala tympani contributes to the outcome. The lack of tools to precisely control the final implant position causes outcome variability, which, along with residual hearing loss, is a major barrier to adoption amongst the current eligible population.

Measurable features collected during implantation can predict outcomes of the surgery. Such features include the cochlear implant placement, insertion force, and structural damage. Currently, these features are sensed qualitatively by the surgeon and the surgeon can adjust the insertion, based on the sensed features. Expert surgeons have optimized the insertion technique to reduce trauma and preserve hearing based on subtle changes as the electrode array is implanted. Currently however, the surgeon's feedback of the insertion force is limited to the resistance they perceive as they manually thread the electrode array into the cochlea, which is limited to the sensitivity of human perception and is highly dependent on the surgeon's experience and dexterity.

Force-measurement systems in robotic platforms have been used to monitor external insertion force during surgery, but only measure the cumulative force and are unable to localize causes of increased insertion force. Additionally, while prior work has attempted to utilize MEMS technology to replace the cochlear implant electrode array, sensing capabilities using dedicated sensors integrated into the electrode array are unknown in the art. There have also been attempts to enhance the surgeon's ability to actuate the electrode array precisely, either using robotically guided insertion, magnetic guidance systems, or built-in actuators. However, these techniques do not incorporate in situ feedback from the cochlear implant electrode array.

### SUMMARY

Described herein is a novel design of an instrumented cochlear implant, wherein the electrode array portion of the implant is provided with one or more sensors to detect various features of the electrode array during insertion and to provide feedback to the surgeon during implantation. The sensor uses a sensor array to collect intraoperative information on the state of the electrode array during insertion. For example, if configured with an array of strain sensors, flexing of the electrode array can be detected at any point along the length of the electrode array. This allows for reconstruction of the pose of the electrode array during insertion and detection of contact or possible contact with the inner walls of the cochlea.

Also disclosed herein is a system for interpreting the signals received from the sensors and providing intraoperative feedback to the surgeon. The signals are digitized and processed via a readout circuit and microcontroller system. The information is presented to the surgeon in real-time via a user interface system such as to allow the surgeon to modify the implantation technique to prevent or minimize trauma to the cochlea.

After implantation, the one or more sensors may be electrically disconnected and left in place.

The system is able to disambiguate multiple causes of increased insertion force (e.g., friction in the scala tympani vs. narrowing of the apex) as well as to provide previously inaccessible data, such as the degree of bending in the electrode array.

### BRIEF DESCRIPTION OF THE DRAWINGS

By way of example, a specific exemplary embodiment of the disclosed system and method will now be described, with reference to the accompanying drawings, in which:

FIG. 1 is a schematic representation of a middle ear, showing positioning of a cochlear implant, and, in particular, the electrode array portion of the implant.

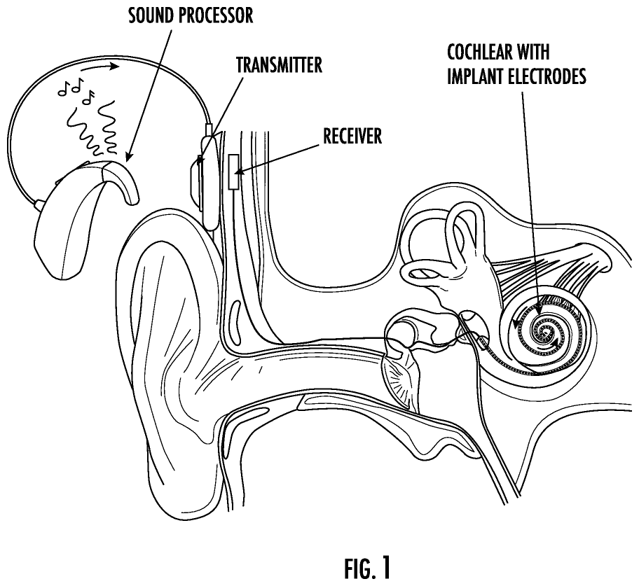

FIG. 2 is a schematic representation of an electrode array having an integrated sensing array.

FIG. 3 is a block diagram of the smart sensing system utilized with the cochlear implant/sensing array of FIG. 2.

FIG. 4A is a schematic representation of a strain sensor of the type that could be used in the embodiments disclosed herein, in a neutral position; FIG. 4B is a schematic representation of the strain sensor in an elongated position.

### DETAILED DESCRIPTION

Disclosed herein is an instrumented electrode array of a cochlear implant wherein the electrode array is configured with one or more sensing elements formed into a microfabricated thin-film sensing array. The sensing elements are preferably microfabricated as thin-film sensors and integrated with the electrode array. Various types of sensors may be deployed as part of the sensing array, including, for example, strain sensors (i.e., resistive, capacitive, or crack-based), force/pressure sensors (capacitive or electrochemical diaphragm), temperature sensors, proximity sensors, optical sensors, optical spectrometry, reflectometry, imaging, coherence tomography based on integrated optical fibers or waveguides, chemical detection, etc. The sensing array may also integrate microfluidic capabilities to enable sensing or to aid surgery via drug delivery or to relieve fluid pressure in the scala tympani.

In various embodiments, one or more different types of sensing elements may be deployed as part of the thin-film sensing array to provide multiple sensing modalities within a single sensing array. Additionally, like sensing elements may be oriented differently on the thin-film. For example, a plurality of strain sensing elements may be oriented in different directions on the thin-film such as to be capable of detecting elongation or compression along multiple axes.

The invention is described herein the context of a microfabricated interdigitated electrode array used as a strain sensor, however, as would be realized by one of skill in the art, any type of sensor previously mentioned or known in the art is contemplated be within the scope of the invention.

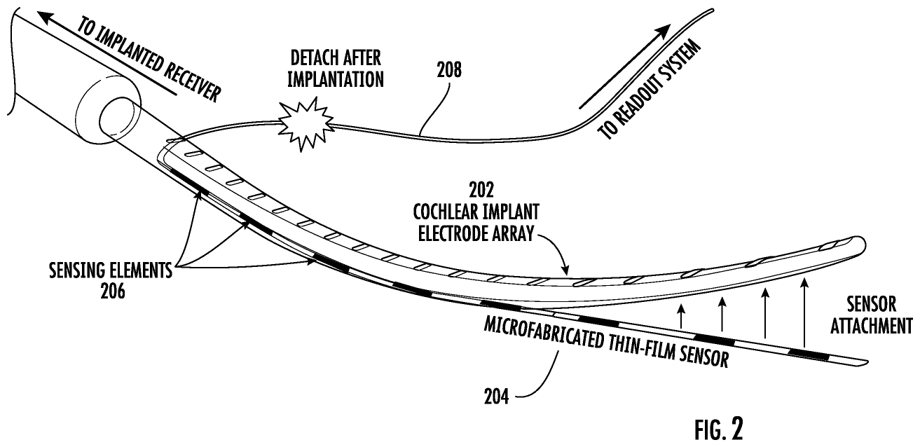

FIG. 2 is a schematic of a specific instantiation of the thin-film microfabricated sensor array 204 designed to attach to or integrate with a cochlear implant electrode array 202 and communicate with a readout system (described below) via flexible cable 208. Microfabricated sensor array 204 comprises a plurality of sensing elements 206 deployed along a length of the array. The actual number of sensors 206 deployed as part of the microfabricated sensor array 204 is dependent on the sensing modality and the desired features to be extracted from the data. For example, in one embodiment wherein the sensing elements 206 are strain sensors, one sensing element 206 may be deployed between each pair of electrodes in electrode array 202 such as to detect flexing of the electrode array 206 along any part of its length.

In one embodiment, the microfabricated thin-film sensor 304 is designed to be disconnected from the readout system after implantation by severing cable 208 as shown in FIG. 2, thereby leaving the inert sensor implanted. In other embodiments, the sensor array 204 may remain active to perform post-operative monitoring, or be removed following surgery.

In one embodiment, the microfabricated thin-film sensing array 204 may be attached to a cochlear implant electrode array 202 after manufacturing via an assembly process. For example, sensor array 204 may be joined to the electrode array 202 using a silicone adhesive. In an alternative embodiment, sensing array 204 may be integrated into the manufacturing process of electrode array 202 by including it, for example, in an injection molding process used to produce electrode array 202. The dimensions of the thin-film sensing array 204 may be varied to match the dimensions of various cochlear implant electrode arrays 202 from different manufacturers.

The construction of the thin-film sensing array 204 is not limited to a single material platform. The one embodiment, the sensing elements 206 use platinum traces embedded in a Parylene C insulation to form an interdigitated electrode array strain sensor. These materials are largely equivalent to other common biocompatible materials such as aluminum and gold to form traces and other polymer insulators, for example, Parylenes, Siloxanes, Polyamide, SU-8, etc. Similarly, an optical waveguide may be implemented with a Parylene C core and silicone cladding (e.g., Parylene photonics), but may also be composed of other materials (e.g., SU-8, Ormocers, etc.).

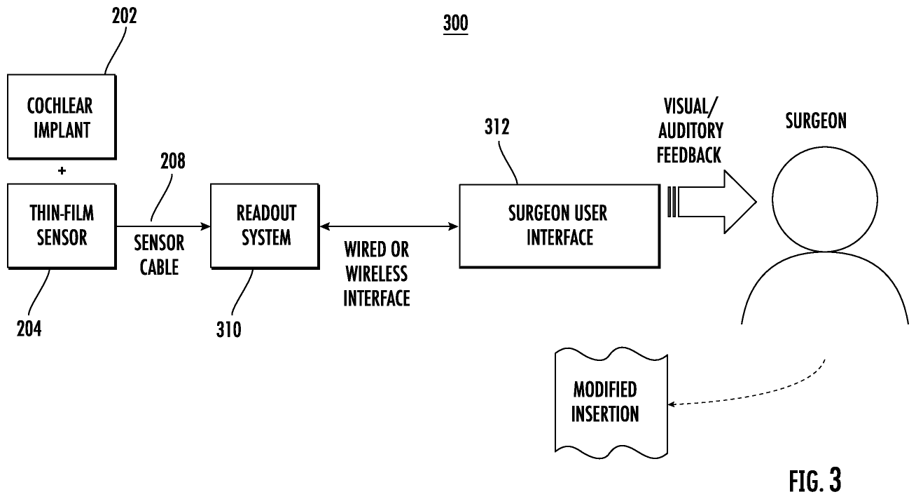

FIG. 3 is a block diagram of a smart sensor system 300 disclosed as a second aspect of the invention. Smart sensor system 300 is composed of multiple components: an electrode array of a cochlear implant 202, a microfabricated thin-film sensing array 204, a readout system 310 which digitizes and processes the signals received from thin-film sensing array 204 via sensor cable 208, and a surgeon (user) interface 312. In various embodiments, system 300 may be stand-alone or integrated into a larger surgical system, for example, a robotically-assisted surgical system. Various systems are also known wherein intraoperative feedback may be provided by the electrodes in the electrode array. In various embodiments, the microfabricated thin-film sensing array 204 described herein and integrated with electrode array 202 may be used in conjunction with or independently of any sensed information collected from the electrodes in electrode array 202.

The microfabricated thin-film sensing array 204 may be as previously described and may utilize one or more optical, electrical, electrochemical or microfluidic systems. One exemplary embodiment of the thin-film sensing array 204 is a metal strain gauge based on an interdigitated electrode array capacitive strain sensor. A second exemplary embodiment of the sensing array 204 is an integrated photonic waveguide to perform fiber optical coherence tomography intraoperatively.

The readout system 310 is composed of several discrete components, preferably integrated on a printed circuit board. Readout system 310 may include any required input/output interfaces, an amplifier and digitizer circuits that may be required to operate the thin-film sensor array 204, including, but not limited to: resistive, capacitive, or impedance measurement circuits, voltage or current sources for electrical sensors, or laser diodes, spectrometers, optical filters, and power meters for optical systems. The readout system 310 also contains a microcontroller to process and store the data, as well as power control (voltage regulators or battery circuitry) and wired or wireless communication circuitry.

The user (surgeon) interface 312 provides feedback to the surgeon and displays the information acquired by the readout system 310 to the surgeon. The feedback and display may consist of audible cues and/or a visual display of metrics (e.g., wrapping factor or tip force), or a more complex visualization (e.g., a 3D pose of the cochlear implant electrode array, or the strain or force distribution along the array). User interface 312 may consist of a device with a screen or speakers, haptic feedback, or an augmented-reality display.

In one embodiment, one or more interdigitated electrode array (IDE) capacitive strain sensors may be utilized as sensing elements 206 on the thin-film sensing array 204. The sensing elements 206 and the overall thin-film sensing array 204 may be fabricated as described in co-pending PCT Patent Application No. PCT/US23/16516 and in Provisional Patent Application No. 63/324,839, to which this application claims priority. The contents of these applications are incorporated herein in their entireties.

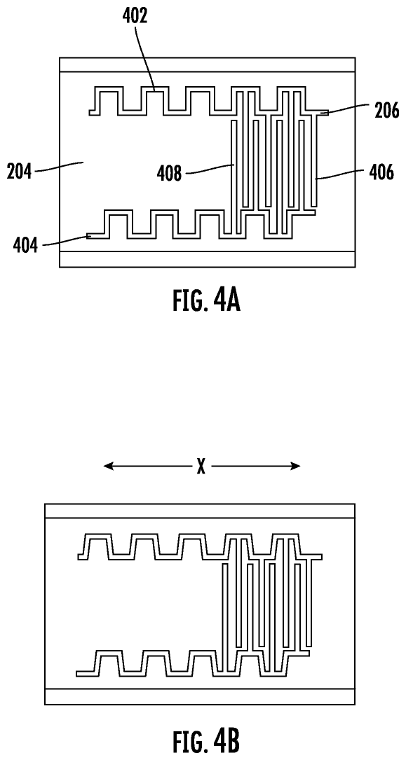

FIGS. 4A,4B are schematic representations of an exemplary strain sensor of the type which may be used as sensing element 206. FIG. 4A shows sensing element 206 in a neutral position, while FIG. 4B is a schematic representation of sensing element 206 in an elongated position. Sensing element 206 comprises a first trace 402 electrically-coupled to a first sub-plurality of the fingers 406 and a second trace 404 electrically-coupled to a second sub-plurality of the fingers 408, wherein the first and second sub-pluralities are exclusive of each other. In embodiments disclosed herein one trace is a ground trace and the other trace is a sense trace. Preferably, fingers in the first sub-plurality will be disposed between two fingers in the second plurality (except at the ends of the array) and vise-versa, thus forming a set of interdigitated fingers. Each of fingers 406, 408 may comprise a stack consisting of a layer of polymer, for example, Parylene C and a thin-film electrically-conductive material, for example, platinum or gold. The polymer layer supporting each finger allows the elongation of the overall device along longitudinal axis X, while still allowing the device to be fabricated using high-volume MEMS fabrication techniques. Fingers 406, 408 may be encapsulated in a protective layer comprised of, for example, PDMS. Traces 402, 404 make use of in-plane trace routing to reduce the stiffness of the sensor along the longitudinal axis of elongation (“X”).

The invention is contemplated to include both the instrumented cochlear implant and the readout and feedback system for providing intraoperative feedback to the surgeon. As would be realized by one of skill in the art, many variations on the system and the device disclosed herein are possible and are contemplated to be within the scope of the invention, which is defined by the claims which follow.

1. A device comprising:
an electrode array; and
a thin-film sensing array comprising one or more sensing elements, integrated with the electrode array; and
a connection coupled to the sensing array for receiving sensing information from the sensing array.

2. The device of claim 1 wherein the electrode array is a component of a cochlear implant and further wherein the electrode array comprises a plurality of electrodes embedded in a substrate.

3. The device of claim 1 wherein the one or more sensing elements are optical or electrical.

4. The device of claim 3 wherein the sensing array further comprises:
one or more electrochemical or microfluidic systems integrated into the sensing array.

5. The device of claim 1 wherein the sensing array comprises one or more sensors of different sensing modalities.

6. The device of claim 3 wherein the one or more sensing elements include strain sensors.

7. The device of claim 6 wherein the strain sensors comprise a plurality of interdigitated fingers.

8. The device of claim 7 wherein each interdigitated finger comprises a stack consisting of a polymer supporting a thin-film conductive later.

9. The device of claim 8 wherein each strain sensor further comprises:
a first trace electrically-coupled to a first sub-plurality of the fingers; and
a second trace electrically-coupled to a second sub-plurality of the fingers;
wherein the first and second sub-pluralities are exclusive of each other.

10. The device of claim 10 wherein the first and second traces are electrically coupled to the electrical connection.

11. The device of claim 1 wherein the thin-film sensing array is adhered to the electrode array using an adhesive material.

12. The device of claim 1 wherein the thin-film sensing array is integrated with the electrode array during a manufacturing process of the electrode array.

13. A system comprising:
an instrumented electrode array;
a readout system, electrically coupled to the electrode array; and
a user interface in communication with the readout system.

14. The system of claim 13 wherein the electrode array is a component of a cochlear implant comprising a plurality of electrodes embedded in a substrate.

15. The system of claim 13 wherein the sensing array comprises one or more sensors of one or more sensing modalities.

16. The system of claim 13 wherein the readout system comprises:
a microcontroller to process and store the data collected from the sensing array; and
circuitry required to operate the sensing array.

17. The system of claim 16 wherein the circuitry comprises:
one or more of resistive, capacitive, or impedance measurement; and
voltage or current sources for powering the sensing elements.

18. The system of claim 14 wherein the user interface comprises:
means for providing audible, tactile, or visual feedback to the user regarding positioning of the electrode array during insertion into a cochlea.

19. The system of claim 18 wherein the visual feedback includes a visualization comprising a 3D pose of the electrode array and/or the strain or force distribution along the electrode array.

20. The system of claim 13 wherein the system is integrated into a robotically-assisted surgical system.

21. The system of claim 13 wherein the robotically-assisted surgical system obtains information regarding the electrode array from the electrodes in the electrode array and further wherein the robotically-assisted surgical system uses both the information from the electrodes and information from the sensing array to provide feedback to the user.
